(19) United States
(12) Patent Application Publication
(10) Pub. No.: US 2023/0210675 A1
(43) Pub. Date: Jul. 6, 2023

(54) SYSTEM AND METHOD FOR AN ARTICULATING ANKLE

(71) Applicant: OTTO BOCK HEALTHCARE LP, Austin, TX (US)

(72) Inventors: Jesse DAY, Holladay, UT (US); Clifton Christensen, Salt Lake City, UT (US); Oleg Pianykh, Salt Lake City, UT (US); Vijay Morampudi, Salt Lake City, UT (US); Herman Boiten, Ede (NL)

(73) Assignee: OTTO BOCK HEALTHCARE LP, Austin, TX (US)

(21) Appl. No.: 18/092,134

(22) Filed: Dec. 30, 2022

Related U.S. Application Data
(60) Provisional application No. 63/295,734, filed on Dec. 31, 2021.

(51) Int. Cl. A61F 2/66; A61F 2/74

(52) U.S. Cl. CPC A61F 2/6607; A61F 2/74; A61F 2002/5006

(57) ABSTRACT

A hydraulic ankle assembly to be used by a user, wherein the ankle assembly includes a base configured to be attached to a spring assembly, a hydraulic cylinder rotatably attached to the base and configured to dampen rotation of the hydraulic ankle assembly, and a prosthetic adapter portion rotatably attached to the base and the hydraulic cylinder and configured to be attached to a prosthetic worn by the user. The base, the hydraulic cylinder, and the prosthetic adapter portion define a force triangle that defines an axis of rotation of the hydraulic ankle assembly. The axis of rotation is positioned in line with the center of mass of the user when the user is standing. The force triangle reduces forces on the hydraulic cylinder.

### CROSS-REFERENCE TO RELATED APPLICATION

This application claims the benefit of U.S. Provisional Patent Application No. 63/295,734, filed Dec. 31, 2021, which is incorporated by reference herein in its entirety.

### TECHNICAL FIELD

The present disclosure relates generally to prosthetic devices, and more particularly relates to prosthetic feet and adjustment features for prosthetic feet.

### BACKGROUND

Amputees are typically fitted with prosthetic devices that meet specific criteria for that particular amputee. For example, the size, shape, stiffness, and other properties of a prosthetic device are selected and tuned to match the size, shape, strength, and other physical properties and functionality of the given amputee. Changes in these properties of a given amputee may influence whether or not a particular prosthetic device will perform properly and according to expectations for the amputee. It is common for an amputee to change his/her prosthetic device when, for example, the amputee grows in height, weight, strength, or balance capability.

Opportunities exist for providing prosthetic devices for amputees that account for changes in the amputee's body and capabilities.

### SUMMARY

One aspect of the present disclosure relates to an ankle assembly. The ankle assembly includes a base configured to be attached to a spring assembly, an extendable link rotatably attached to the base and configured to control rotation of the ankle assembly, and a prosthetic adapter portion rotatably attached to the base and the extendable link and configured to be attached to a prosthetic worn by the user. The base, the extendable link, and the prosthetic adapter portion define a force triangle that defines an axis of rotation of the ankle assembly. The axis of rotation is positioned below and in line with the center of mass of the user when the ankle assembly is attached to the user.

Another aspect of the present disclosure relates to a ankle assembly including a prosthetic adapter portion, a extendable link rotatably attached to the prosthetic adapter portion to define a first pivot point, a base attached to the prosthetic adapter portion to define a second pivot point, and a third pivot point defined by a rotational attachment between the extendable link and the base. The extendable link has a translational axis of extension and compression which allows the extendable link to change length. The extendable link may be comprised of a hydraulic cylinder. The hydraulic cylinder is configured to dampen rotation of the hydraulic ankle assembly. The first pivot point and the second pivot point define a first pivot distance, the second pivot point and the third pivot point define a second pivot distance, and the first pivot point and the third pivot point define a third pivot distance. The first pivot distance is greater than 30 millimeters (mm), the second pivot distance is greater than 30 mm, and the third pivot distance is greater than 25 mm. The third pivot distance defines a moment arm of the hydraulic cylinder about the second pivot.

A further aspect of the present disclosure relates to a prosthetic foot system which includes a spring assembly having a toe end portion and a heel end portion and a hydraulic ankle assembly attached to the spring assembly. The hydraulic ankle assembly includes a base attached to a spring assembly, a hydraulic cylinder rotatably attached to the base and configured to dampen rotation of the hydraulic ankle assembly, and a prosthetic adapter portion rotatably attached to the base and the hydraulic cylinder and configured to be attached to a prosthetic worn by the user. The base, the hydraulic cylinder, and the prosthetic adapter portion define a force triangle that defines an axis of rotation of the spring assembly and base about the prosthetic adapter portion. The axis of rotation is positioned below and in line with the center of mass of the user when the user is in a standing position. The location of the axis of rotation minimizes the force exerted on the hydraulic cylinder when the user is standing, which increases standing stability.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a perspective view of an exemplary prosthetic foot system including a footshell and a prosthetic foot in accordance with the present disclosure.

FIG. 2 is a perspective view of the prosthetic foot system of FIG. 1 including a spring assembly and a hydraulic ankle assembly.

FIG. 3 is another perspective view of the prosthetic foot of FIG. 1.

FIG. 4 is another perspective view of the prosthetic foot of FIG. 1.

FIG. 5 is a perspective view of the prosthetic foot of FIG. 1 with an exploded view of the hydraulic ankle assembly.

FIG. 6 is a perspective view of a hydraulic cylinder of FIG. 5.

FIG. 7 is another perspective view of a hydraulic cylinder of FIG. 5.

FIG. 8 is an exploded view of the hydraulic cylinder of FIG. 5.

FIG. 9 is another exploded view of the hydraulic cylinder of FIG. 5.

FIG. 10 is a top view of the hydraulic cylinder of FIG. 5.

FIG. 11 is a cross-sectional view of the hydraulic cylinder shown in FIG. 10, taken along cross-section indicators A-A.

FIG. 12 is a cross-sectional view of the hydraulic cylinder shown in FIG. 10, taken along cross-section indicators B-B.

FIG. 13 is a cross-sectional view of the hydraulic cylinder shown in FIG. 10, taken along cross-section indicators C-C.

FIG. 14 is a cross-sectional view of the hydraulic cylinder shown in FIG. 10.

FIGS. 15A and 15B are cross-sectional views of the hydraulic cylinder shown in FIG. 10.

FIG. 16 is a cross-sectional view of the hydraulic cylinder shown in FIG. 2.

FIG. 17 is a cross-sectional view of the hydraulic cylinder shown in FIG. 10.

FIG. 18 is a cross-sectional view of the hydraulic cylinder shown in FIG. 10.

FIG. 19 is a cross-sectional view of the hydraulic ankle assembly shown in FIG. 5.

FIG. 20 is a hydraulic diagram of the hydraulic cylinder shown in FIG. 10.

FIG. 21 is a side view of an embodiment of the hydraulic cylinder shown in FIG. 10 in accordance with the present disclosure.

FIG. 22 is a side view of an embodiment of the hydraulic cylinder shown in FIG. 10 in accordance with the present disclosure.

FIG. 23 is a side view of an embodiment of the hydraulic cylinder shown in FIG. 10 in accordance with the present disclosure.

FIG. 24 is a cross-sectional view of the prosthetic foot system shown in FIG. 1.

FIG. 25 is another cross-sectional view of the prosthetic foot system shown in FIG. 1.

FIG. 26 is another cross-sectional view of the prosthetic foot system shown in FIG. 1.

FIG. 27 is another cross-sectional view of the prosthetic foot system shown in FIG. 1.

FIG. 28 is another cross-sectional view of the prosthetic foot system shown in FIG. 1.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

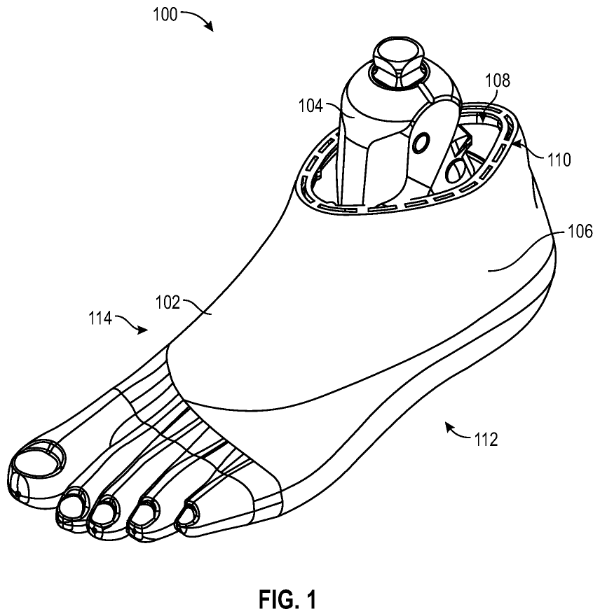

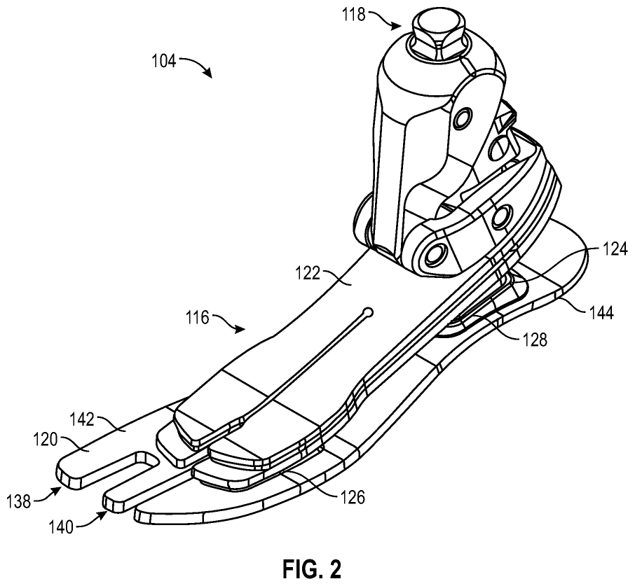

FIG. 1 illustrates a perspective view of an example of a prosthetic foot system 100 including a footshell 102 and a prosthetic foot 104. FIG. 2 illustrates a schematic cross section of the prosthetic foot system 100. Referring now to FIGS. 1 and 2, the example prosthetic foot system 100 is shown and described. The prosthetic foot system 100 includes the footshell 102 and the prosthetic foot 104. In some embodiments, the footshell 102 may be formed as a unitary device without any separate components. In other embodiments, the footshell 102 and the prosthetic foot 104 may be formed as separate components and assembled after the footshell 102 and the prosthetic foot 104 are formed. The prosthetic foot 104 is configured to support the user of the prosthetic foot system 100 and the footshell 102 is configured to house and protect the prosthetic foot 104 and to have an aesthetic design. The footshell 102 provides an aesthetic covering for the prosthetic foot 104 to give the appearance of an actual foot. The prosthetic foot 104 is intended to be used inside a shoe. The prosthetic foot system 100 is configured to be mounted to a limb (not shown), such as a residual limb remaining after an amputation. An example of a residual limb may be a residual limb associated with a below-the-knee amputation. One or more of the footshell 102 and the prosthetic foot 104 may be formed or manufactured via an additive manufacturing process such as 3D printing, which forms the footshell 102 and/or the prosthetic foot 104 from a three-dimensional lattice network.

Lower limb prosthetic components may benefit from adjustability and are typically connected using an industry standard prosthetic pyramid connection. A prosthetic pyramid connection typically consists of a male pyramid connector/adapter and a complementary female pyramid connector/adapter connected to each other. The combination of the male and female adapters may provide for angular adjustment between two prosthetic components. The male portion may include two primary features: a pyramid protrusion and a contoured (e.g., spherical) surface. The pyramid protrusion may have four planar surfaces that are oriented in posterior, anterior, medial, and lateral directions. These surfaces may be angled with respect to a pyramid axis, wherein the pyramid axis extends along a longitudinal axis of the shin or thigh of the limb. The pyramid surfaces typically are angled in the range of about 10 degrees to about 30 degrees, and more particularly about 15 degrees. Due to the angles of the four pyramid surfaces, the protrusion necks down in a distal direction. The necked down end transitions to the contoured (e.g., spherical) surface. The contoured portion may be part of a separate base component with the pyramid protrusion fixedly and rigidly attached to the base component. Alternatively, the base component may be integrated to the spherical feature where the two features are combined into a single monolithic block of material.

The pyramid protrusion may be threaded into the base component with the threads glued or otherwise fixed to prevent unthreading. Alternate methods of fixedly attaching a pyramid protrusion to a base component including a spherical surface are possible such as, for example, creating a stud on the narrow end of the pyramid protrusion and molding the stud into a fiber reinforced moldable base material, or by deforming the stud such that the stud creates a strong interference fit between the a pyramid protrusion and the base component. A male pyramid adapter may be monolithic meaning it is formed or composed of a single, continuous material without joints or seams. However, as discussed herein, the male pyramid adapter may comprise a plurality of components, such as one or more components that adjust an effective length of the pyramid adapter.

A female pyramid adapter may include a predominantly hollow cylinder with a spherical surface formed on one end and four threaded fasteners. The inner surface of the cylinder may not be round or cylindrical as recesses are commonly formed on this surface to allow increased articulation of the male protrusion within the cylinder while adjusting the angle between the components. The spherical surfaces of both the male and female components have a near identical spherical radius to allow mating with each other. Fasteners (e.g., two or more fasteners) may be threaded into the cylinder at an angle relative to the cylinder axis (e.g., 15 degree angle), and the fasteners may engage one or more of the four planar surfaces of the male pyramid protrusion to releasably secure the position of the pyramid connection. By adjusting the depth of the fasteners in the female component, the angle between the male and female pyramid components can be changed and the angle between two prosthetic components can be adjusted. A female pyramid component may be referred to as a pyramid receiver. A female pyramid adapter may be monolithic. The threaded fasteners typically are separate components in a monolithic female pyramid adapter.

A male or female pyramid adapter component may be machined or formed directly onto a prosthetic device, for example, a prosthetic knee. For the purposes of this disclosure, a pyramid adapter may be either the male or female component of a pyramid connection and include either a pyramid protrusion and a spherical mating surface in the case of a male pyramid adapter or a spherical mating surface with multiple (e.g., four) threaded fasteners to engage and lock a pyramid protrusion in the case of a female adapter. A pyramid adapter may be fabricated separately from other components and include design features allowing the adapter to be attached to other prosthetic components in addition to connecting the complementary opposite component of a pyramid connection.

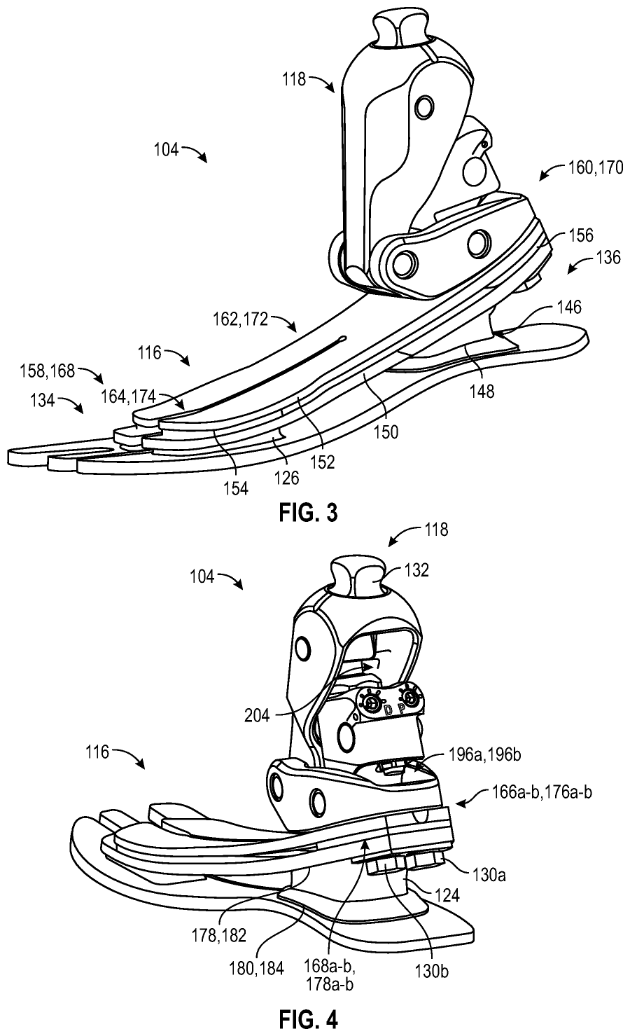

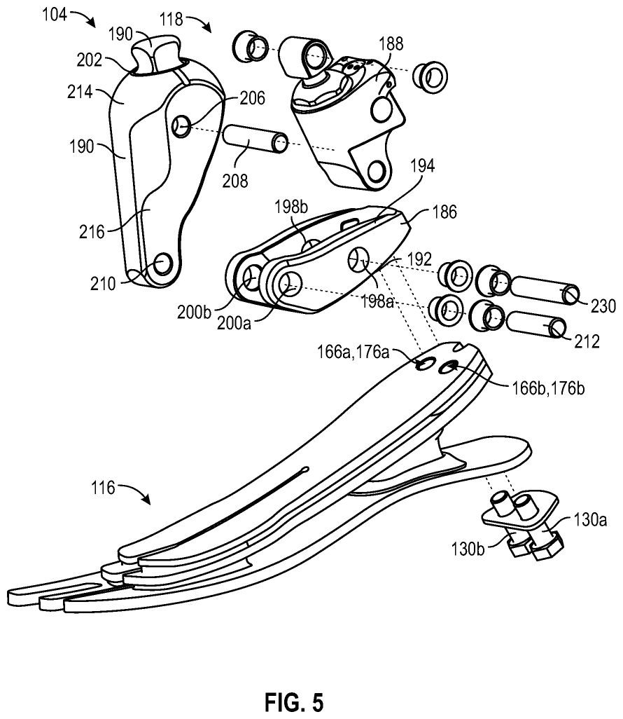

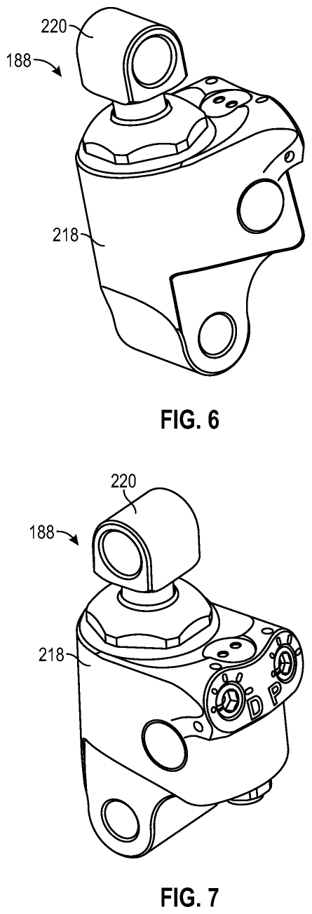

FIG. 2 illustrates a perspective view of the prosthetic foot 104. FIG. 3 illustrates another perspective view of the prosthetic foot 104. FIG. 4 illustrates another perspective view of the prosthetic foot 104. FIG. 5 illustrates an exploded perspective view of the prosthetic foot 104. Referring now to FIGS. 2, 3 and 4, the prosthetic foot 104 is shown including a spring assembly 116 and an hydraulic ankle assembly 118. The spring assembly 116 includes a base spring 120, a top spring assembly 122, and a heel cushion 124. The spring assembly is rigidly and fixedly attached base 192. The top spring assembly 122 is connected to the base spring 120 in a toe end area at a toe end connection 126. The toe end connection 126 may include a bond connection formed by, for example, an adhesive bond. The toe end connection 126 may be formed using an elastic, flexible material that provides at least some relative movement between the base spring 120 and top spring assembly 122 (e.g., rotational movement about a vertical axis, compression, and translational movement in the anterior/posterior and/or medial/lateral direction). The toe end connection 126 may provide the sole connection point between the base spring 120 and top spring assembly 122. Typically, the heel cushion 124 is mounted directly to a top surface of the base spring 120 and arranged to contact a bottom surface of the top spring assembly 122. The heel cushion 124 may be releasably connected to the base spring 120. Alternatively, heel cushion 124 may be releasably connected to the top spring assembly 122. In at least some examples, the heel cushion 124 is connected to the base spring 120 with an interference fit connection using, for example, a retainer 128 that is mounted to the top surface of the base spring 120. The heel cushion 124 may be replaceable with other heel cushions having different properties such as increased or reduced stiffness, compressibility, damping capability, etc. Heel cushions of different sizes and shapes may also be used in place of the heel cushion 124 shown in the figures. In some examples, the prosthetic foot 104 may be operable without any heel cushion 124.

Referring to FIGS. 3, 4, and 5, the hydraulic ankle assembly 118 may be releasably attached to the top spring assembly 122 at its proximal end. In at least one example, the hydraulic ankle assembly 118 is releasably connected using one or more fasteners 130a, 130b. A prosthetic adapter portion with different connector features such as a pyramid connector 132 may be used, for example, a female pyramid adapter may replace the male pyramid adapter 132. In at least some examples, the pyramid connector 132 is a replaceable component of the hydraulic ankle assembly 118. In other embodiments, the pyramid connector 132 is integrally formed with remaining portions of the adapter assembly. Other connector features besides a pyramid connector may be used as part of the adapter assembly for securing the prosthetic foot 104 to another prosthetic member such as a lower leg pylon, a socket, or the like.

The base spring 120 is shown including a toe end 134, a heel end 136, a sandal slot 138, and a balance slot 140. The base spring 120 may also include a top surface 142, a bottom surface 144, and the heel cushion retainer 128 positioned at a heel end portion of the base spring 120. The retainer 128 may include a cavity 146 and a rim 148 to help releasably secure the heel cushion 124 to the base spring 120.

The sandal slot 138 may also have a length Ls. The length of the sandal slot 138 is typically in the range of about 0.5 to about 2 inches. The sandal slot 138 is formed in the toe end portion of the base spring 120 and extends posterior from an interior most edge of the base spring 120. The balance slot 140 is also formed at the toe end portion beginning at the anterior most edge of the base spring 120 and extending posteriorly. In at least some embodiments, the balance slot 140 is aligned with a longitudinal center line of the base spring 120. The balance slot 140 may provide enhanced medial/lateral compliance for the prosthetic foot 104, particularly when walking on uneven surfaces.

As shown in at least FIGS. 2-4, the base spring 120 has a contoured shape along its length. The side profile of the base spring 120 undulates between concave and convex shapes. In some examples, the distal surface of the base spring 120 is preferably convex in an anterior section, transitions to concave in an arch or mid-section, and may transition back to convex at the posterior end. These contours and the location of the contours, particularly relative to the toe end connection 126 and the heel cushion 124, may provide improved rollover smoothness, enhanced energy feedback to the user, stability, and comfort during use of the prosthetic foot. Providing the lever portion extending posterior of the heel cushion 124 may also provide improved smoothness in the rollover and energy feedback during use.

The top spring assembly 122 is shown including first and second spring members 150, 152, a first spacer 154 at the toe end portion of the prosthetic foot, a second spacer 156 positioned at a proximal end of the top spring assembly 122 and a gap G provided between the first and second spring members 150, 152 along their entire length. The first and second spring members 150, 152 may be referred to as leaf springs. The first and second spring members 150, 152 may extend generally in parallel with each other along their entire lengths. The first spacer 154 may be provided as a bond connection between the first and second spring members 150, 152. In at least some examples, the first spacer 154 comprises the same bond material as used for the toe end connection 126 between the top spring assembly 122 and the base spring 120. In at least some embodiments, the first spacer 154 is positioned generally in alignment with the toe end connection 126 so as to be positioned vertically above the toe end connection 126, or at least partially overlapping the toe end connection 126 in a length dimension of the base spring 120. The first spacer 154 may provide a permanent connection between the first and second spring members 150, 152. The material of first spacer 154 may provide at least some relative movement between the first and second spring members 150, 152 (i.e., rotational movement about a vertical axis, translational movement in an anterior, posterior or medial/lateral direction, compression, etc.). The material of first spacer 154 may be elastic so as to return to its original shape upon removal of a force that is used to compress or deform the first spacer 154.

In other examples, the first spacer may comprise a wear resistant, low friction material that is attached to one of the first and second springs. The first spacer is not attached or connected to the other of the first and second springs. This arrangement supports compression forces between the distal ends of the first and second springs and allows the springs to separate during plantar flexion and also slide against each other at the distal ends of the springs. Such an embodiment may also alter performance of the foot during rollover in comparison to having the first spacer as a bond connection. Tensile and shear forces are not transferred through the spacer, hence the deflection and stress conditions in the upper spring assembly are modified. The first spring is in an unloaded condition during plantarflexion at heel strike and, as the foot rolls over and the user's weight is transferred to the toe, shear displacement between the distal ends of the first and second springs results in increased defection the foot in the toe region, thereby softening the foot during both the heel strike and terminal stance portions of the gait cycle

The second spacer 156 may comprise a rigid material that is non-compressible and/or non-elastic. The second spacer 156 may be positioned at a proximal most end of the top spring assembly 122. The second spacer 156 may be aligned with the hydraulic ankle assembly 118, or at least portions thereof. In the illustrated embodiment, the second spacer 156 includes apertures through which the fasteners 130a, 130b extend for connection of the hydraulic ankle assembly 118 to the top spring assembly 122.

The first and second spacers 154, 156 may define the size of the gap G when the prosthetic foot 104 is in a rest state. Typically, the gap G is provided along an entire length of the first and second spring members 150, 152 when the prosthetic foot 104 is in a rest state (i.e., prior to application of a force during use of the prosthetic foot 104). Alternatively, the two upper springs 150, 152 may abut (e.g., directly contact each other) at the connector location. The gap G may vary in size during operation of the prosthetic foot 104. For example, the gap G may reduce in size at the first spacer 154 if the material of the first spacer 154 is compressible during use. In another example, the gap G may reduce or change size at locations between the first and second spacers 154, 156 during use. For example, applying a force from a user during a gait cycle may change the size of gap G at various phases of the gait cycle (e.g., at heel strike, stance phase, and toe off), as the forces are applied and released during use by a wearer, those forces are absorbed and/or fed back through the base spring 120 and heel cushion 124. In at least some embodiments, the first spring member 150 may come into contact with the second spring member 152 during use of the prosthetic foot (i.e., the gap reduces to zero).

The first spring member 150 is shown having an anterior end 158, a proximal end 160, a horizontal portion 162, a slot 164, and fastener apertures 166a, 166b. The second spring member 152 may include an anterior end 168, a proximal end 170, a sloped portion 172, a slot 174, and fastener apertures 176a, 176b. The slot 174 may be aligned with the slot 164 of the first spring member 150 and the balance slot 140 formed in base spring 120. In at least some examples, the slots 140, 164, 174 may extend in a posterior direction to a common location. The slots 140, 164, 174 may terminate at different locations in the anterior direction. The slots 164, 174 may be aligned with a center line of the base spring 120 and top spring assembly 122 so as to provide balanced medial/lateral pronation and compliance during use of the prosthetic foot.

The top spring assembly 122 is mounted to the base spring 120 as shown in at least FIGS. 2-4. The heel cushion 124 is arranged to contact a bottom or downward facing side or surface of the top spring assembly 122 (e.g., a bottom surface of first spring member 150 as shown in FIG. 4). Although the heel cushion 124 is shown connected to the base spring 120 and not the top spring assembly 122, other embodiments may provide the heel cushion 124 connected to both the base spring 120 and top spring assembly 122, or connected only to the top spring assembly 122 (e.g., the retainer 128 is mounted to the bottom surface of first spring member 150 for releasable attachment of the heel cushion 124).

The heel cushion 124 may be releasably mounted to the base spring 120 (or top spring assembly 122). Alternatively, the heel cushion 124 may be permanently connected to the base spring 120. The replaceability of heel cushion 124 may provide customization of the amount of heel stiffness, cushioning, energy dampening, and the like provided by heel cushion 124. Heel cushion 124 may be connected with an interference fit connection. Other embodiments may provide for the heel cushion 124 to be secured with a positive connection such as, for example, a fastener, clip, bracket or the like.

The heel cushion 124 may include a top surface 178 (see FIG. 4), a tapered shape having a variable thickness along its length, a bottom surface 180, and top and bottom perimeter rims 182, 184. The tapered shape may provide for a smaller thickness at an anterior end as compared to a greater thickness at a posterior end of the heel cushion 124, as shown in FIG. 4. The tapered shape of the heel cushion 124 may match the angle and/or curvature of the first spring member 150. As such, the top surface 178 may have a contoured shape rather than a planar shape. Similarly, the bottom surface 180 may have a shape that matches the contour or curvature of the top surface of the base spring 120, as shown in at least FIG. 4.

The heel cushion 124 may comprise a shock absorbing, dampening material such as, for example, silicone or urethane elastomers including, for example, silicone or urethane foams. In some embodiments, the heel cushion 124 may include a plurality of different materials, layers of materials, or separate components that are secured together as an assembly to provide the desired cushioning properties. In one example, the heel cushion 124 includes a foam material encapsulated within a protective polymer shell. In another example, the heel cushion 124 includes a gel material or capsule that is encapsulated within a foam material.

The base spring 120 and first and second spring members 150, 152 may comprise a fiber reinforced composite material such as, for example, carbon fiber reinforced composite. The first spacer 154 may include an adhesive bond comprising a flexible adhesive such as, for example, a urethane adhesive having a Shore A hardness in the range of about 70 to about 95. During manufacture of the top spring assembly 122, the first and second spring members 150, 152 may be bonded together using a removable gasket between the springs to create a sealed space for the adhesive, and the adhesive is then injected into the space.

The second spring member 152 may be shorter in length than the length for the first spring member 150. This difference in length may allow for a somewhat gradual change in stiffness in the top spring assembly 122. Although two spring members 150, 152 are shown as part of the top spring assembly 122, other embodiments may utilize more than two leaf spring elements, and the leaf spring elements may have the same or different lengths.

The second spacer 156 may comprise a lightweight material such as, for example, aluminum, nylon or fiberglass sheet material (e.g., fiberglass G-10). The top spring assembly 122 may provide a connection between the first and second spring members 150, 152 at opposite ends with a gap G provided there between, thereby providing a number of unexpected structural advantages. These advantages in connection with the type of spacers 154, 156, the toe end connection 126, the heel cushion 124, and/or other features may provide a number of performance advantages as compared to known prosthetic feet. For example, a dual, narrow cantilever beam, one located above the other and with a space in between the upper and lower beams, and with frictionless spacer at the free end to transmit an applied vertical force from the upper beam to the lower beam at the free end, may result in about 15-25% reduction in bending stress and about 30-45% reduction in shear stress as compared to an equivalent stiffness single cantilever beam. If the first spacer is comprised of a low friction material connected to one of the first and second springs, the boundary conditions described are highly accurate.

If the first spacer is a bond connection (e.g., created with a flexible material), the boundary conditions are approximately midway between a frictionless spacer between the distal ends of the first and second spring and a rigid connection at the distal ends of the first and second springs. Because stresses are reduced by using the dual upper spring design, a prosthetic foot utilizing this dual spring design exhibits at least one of improved durability and improved flexibility as compared to single spring designs and dual spring designs which are rigidly connected at the distal end. Furthermore, utilizing a low friction spacer material may provide more flexibility than utilizing a flexible bond connection, thus potentially providing opportunities to achieve different and desirable performance characteristics and multiple design options to achieve the designer's goal. Many of the advantages of the dual cantilever beam designs disclosed herein may be maximized when the two beams (e.g., first and second spring members 150, 152) have substantially equal bending stiffness. If the beams are constructed of unidirectional fiber reinforced composite lamina, the maximum strength/stiffness ratio may be best achieved when both beams have substantially the same lamina orientation and thickness. As the difference between the bending stiffness of the upper and lower beams increases, the advantages of a dual cantilever spring design typically diminish.

The heel cushion 124 may comprise a silicone or urethane elastomer (e.g., an elastomer with the Shore hardness range of about 50A to about 90A). The heel cushion 124 may be retained with retainer 128 in a way that extends around an entire perimeter of the heel cushion 124. Other embodiments may provide for a retainer that extends around only a portion of perimeter of the heel cushion 124. The retainer 128 may be bonded to the top surface 142 of the base spring 120 using, for example, an adhesive. In some embodiments, both the adhesive and the retainer 128 are somewhat flexible to avoid detachment of the retainer 128 from the base spring 120 when the base spring 120 flexes during use. The retainer 128 and adhesive may comprise a plastic material having a Shore hardness in the range of, for example, about 90A to about 50D. Alternatively, the retainer 128 may be cast into the structure of base spring 120 along the top surface 142 thereof, which may eliminate the need for use of an adhesive or other bonding agent.

The retainer 128 may help keep the heel cushion 124 in place by utilizing geometric interlocking features. These interlocking features may include angled (e.g., wedge-shaped) features in the retainer and along an exterior of the heel cushion 124, wherein corresponding surfaces interface to provide a connection The heel cushion 124 may be deformed or compressed in order to fit into the interior of the retainer 128, and then expanded automatically to its original shape thereby creating an interference fit connection between the features of the retainer 128 and the heel cushion 124. Alternatively, the retainer 128 and the heel cushion utilize a rib that fits into a recess, wherein the rib and recess may be formed on either the retainer 128 or heel cushion 124.

Generally, the base spring 120 extends from the toe region to a heel region of the prosthetic foot. The base spring 120 may extend from an interior most point of the prosthetic foot to a posterior most point of the prosthetic foot. The top spring assembly 122 may be connected to the base spring 120 at a location spaced posterior of an anterior most edge of the base spring 120. In at least one example, the top spring assembly 122 is positioned posterior of the sandal slot 138 formed at the distal end of the base spring 120. The base spring 120 may extend in an anterior direction at least as far as an anterior most point along a length of the top spring assembly 122.

As discussed above, the slot or split 140 formed in the base spring 120 from the anterior edge in a posterior direction may be aligned with the slots or slits 164, 174 formed in the top spring assembly 122 from the anterior end of the top spring assembly 122 extending in a posterior direction. These slots or splits may provide for the entire prosthetic foot 104 to be divided into medial and lateral sides at least in the toe and midfoot regions of the prosthetic foot.

The top spring assembly 122 includes first and second spring members 150, 152 that extend to different anterior positions along the length of the prosthetic foot. At least FIG. 4 illustrates the first spring member 150 extending further in an anterior direction than the second spring member 152. The first spacer 154 is positioned at the anterior most edge of the second spring member and spaced posterior of the anterior most edge of the first spring member 150.

The top spring assembly 122 extends generally parallel with the base spring 120 in the toe, midfoot, and heel regions of the base spring 120. As described above, other embodiments may provide for the top spring assembly 122 to continue extending in a generally horizontal or slightly angled direction relative to the base spring 120 and/or a horizontal plane through the heel end portion.

Additionally, the gap G may be substantially constant when the prosthetic foot 104 is in a rest or unloaded state. During use of the prosthetic foot 104, portions of the first and second spring members 150, 152 may move toward and/or away from each other to alter the size of gap G at various locations along the length of the top spring assembly 122. In at least some embodiments, portions of the first and second spring members 150, 152 may contact each other.

The fasteners 130a-b may be arranged side-by-side in a medial/lateral direction. In other arrangements, the fasteners 130a-b may be arranged in alignment with a length dimension of the prosthetic foot 104. Although only two fastener 130a-b are shown in FIG. 4, only one or more than two fasteners 130a-b may be used. The fasteners 130a-b may provide a positive connection between the first and second spring members 150, 152, a positive connection between the top spring assembly 122 and the hydraulic ankle assembly 118, and/or a positive connection between one or both of the first and second spring members 150, 152, and the spacer 156. In some examples, the fasteners 130a-b are connected directly to one or both of the first and second spring members 150, 152 (e.g., to a threaded seat formed in one or both of the first and second spring members 150, 152), or may be connected to a nut (not shown) positioned on an opposite side of the top spring assembly 122.

The prosthetic foot 104 may provide energy feedback, stability, force dampening and the like associated with the use of spaced apart spring members in the top spring assembly 122, the use of a heel cushion 124 arranged in the specific location and having the size and shape shown in FIG. 4, the shape and size of the top spring assembly 122 and base spring 120, and the size, shape, and orientation of the hydraulic ankle assembly 118. Furthermore, the base spring 120 and top spring assembly 122 may include slots (e.g., slot 140 for base spring 120 and slots 164, 174 for first and second spring members 150, 152) that provide medial/lateral pronation and ambulation for the prosthetic foot 104, which may provide improved stability for the user, particularly on uneven ground surfaces.

The prosthetic foot 104 may be a dual or multiple toe spring prosthetic foot. The prosthetic foot 104 may be a single toe spring prosthetic feet. The heel assemblies, adapter assemblies, attachment assemblies, and other features disclosed with reference to any single embodiment disclosed herein may be interchangeable with features of other prosthetic foot embodiments disclosed herein.

In alternative embodiments, the connection between the base spring and the top spring assembly and between the first and second spring members in the anterior region of the foot and may be provided with bolts or other fasteners. A rigid spacer may be provided between the spring members and/or between the top spring assembly and the base spring. The use of bolts or other fasteners in combination with an altered geometry of the first and second spring members may eliminate gaps that may otherwise exist at connection points at the anterior end of the prosthetic foot. In another embodiment, a connection between the first and second spring members may be made by wrapping carbon fiber or glass fiber around the first and second spring members at the connection point between the first and second spring members, and securing the spring members and the fiber by impregnating the fiber with epoxy or similar thermosetting resin. A similar connection may be made between the top spring assembly and the base spring.

In another example, the connection at the proximal end of the top spring assembly may be created by altering a geometry of the first and second spring members such that no gap exists at the connection points between the first and second springs. In this arrangement, a gap may still be provided between the first and second spring members at other locations along their lengths. In some embodiments, one or more of the first and second spring members may be inserted into a slot formed in the prosthetic connector (e.g., base 186 of ankle assembly 118), and the first and second spring members are secured together and to the prosthetic connector with an adhesive or a fastener.

The ankle assembly 118 includes a base 186, an extendable link 188, and a prosthetic adapter portion 190 pivotably attached to each other. The extendable link may be comprised of a hydraulic cylinder. The ankle assembly 118 may be a passive hydraulic damping system that provides the ankle joint member damping rotational resistance, with adjustable independent damping resistances in both the plantarflexion and dorsiflexion directions. The ankle assembly 118 provides the user a more natural feel during the gait-cycle. A hydraulic ankle assembly 118 gives the amputee some fluid-like movement during normal use, not a more rigid feel that is associated with the typical prosthetic foot. Specifically, the hydraulic ankle assembly 118 includes: (1) a soft dorsi flexion stop that improves the transition between the hydraulic resistance and spring resistance created by the carbon foot spring elements; (2) a manual hydraulic lock which prevent plantarflexion of the ankle such that when the ankle reaches maximum dorsiflexion it will be locked; (3) a nitrogen-filled accumulator, or volume compensator that maintains the hydraulic system at a preloaded pressure on the fluid and compensates fluid loss; (4) a hydraulic lock that may lock the ankle in any position to enable the user to use different height heels (shoes) with the same prosthetic ankle and foot; and (5) an improved hydraulic geometry by relocating the ankle pivot points such that a high percentage of the axial load is supported by the pivot structure when the user is standing, and the hydraulic cylinder has improved leverage about the base and foot spring pivot point, reducing the pressure of the hydraulic fluid in the system, reducing the required strength and mass of the hydraulic cylinder, and increasing cycle life and seal integrity.

Specifically, the hydraulic ankle assembly 118 enables plantarflexion motion at an ankle joint, which allows the metatarsophalangeal (i.e. ball of foot) area of the foot to achieve contact with the ground earlier in the gait cycle. The ball of foot/wide part of the foot provides stability during the gait cycle. The hydraulic ankle assembly 118 also enables a small amount of dorsiflexion, relative to a standing position, which results in reduced and adjustable resistance to tibial progression when the prosthetic shin is vertical or near vertical when compared to foot without a hydraulic ankle assembly. When an amputees' center of mass is directly above the shin and the shin is vertical, the amputee does not have much leverage over the lever arm created by the forefoot of a prosthetic foot. The hydraulic ankle assembly 118 further enables toe clearance during swing phase which reduces stumbling and falling. Because the rotation around the hydraulic ankle assembly 118 is dampened by hydraulic resistance, the foot portion stays in a dorsiflexed position during swing phase until the heel makes contact with the ground and plantarflexion begins.

For example, as described below, the rotation axis of the foot springs is forward of the pyramid axis. Therefore, the center of mass (COM) of the amputee is directly above the rotation axis when standing, allowing amputees to stand without significant movement of the hydraulic ankle assembly 118. This design feature also minimizes impact when the ankle reaches the end of its hydraulic range in the dorsiflexion direction (the dorsiflexion stop).

The hydraulic ankle assembly 118 also includes a dorsiflexion stop bumper 274 that reduces or eliminates an impact at the end of the dorsiflexion travel. As the hydraulic ankle assembly 118 dorsi flexes, it reaches the end of its hydraulic range-of-motion and the hydraulic action abruptly stops. At this point the flex of the spring assembly 116 takes over and starts to bend. This transition can be abrupt and not comfortable for the amputee. The dorsiflexion stop bumper gradually squeezes as the hydraulic ankle assembly 118 reaches this transition point, allowing a smooth transition from the hydraulic function to the flexing function of the spring assembly 116. Additionally, the dorsiflexion stop bumper 274 may be low-profile disc springs that would respond is a similar way as the elastomeric stop and provide a smooth transition between the hydraulic function and the carbon spring function of the hydraulic ankle assembly 118 and spring assembly 116.

Moreover, the geometry of the hydraulic ankle assembly 118 described herein may include a greater distance between the pivot axes on the base 186 compared to other hydraulic ankle/feet. Increased pivot distance increases the distance between the cylinder axis and the foot pivot point (the moment arm), which reduces the forces on the hydraulic cylinder and results in reduced stresses and strains in the hydraulic cylinder assembly and also allows for either decreased hydraulic pressure or a smaller cylinder and piston diameter at equivalent pressure. Reducing forces and maximum hydraulic pressures increases reliability and reduces weight.

The hydraulic ankle assembly 118 described herein utilizes a straight cylinder including three pivot axes to eliminate transverse loads on the cylinder and cylinder shaft. At least some known hydraulic ankle assemblies may include only two pivot axes, resulting in transverse load on the cylinder and shaft which is accommodated with large clearances between parts which may result in fluid leakage as seals wear. At least some known hydraulic ankle assemblies may include an extra link added the end of the shaft to achieve three pivot axes, increasing the part count, weight, and cost. The three pivot axes design, without an additional link, disclosed in the hydraulic ankle assembly 118 is an improvement over current designs.

At least some known passive hydraulic systems may not be locked in the dorsi flexion position. The limiting range of motion in current designs occur when the hydraulic piston reaches its maximum position in the cylinder. Once the piston reaches this maximum position (whether in the dorsi flexion or plantar flexion positions) it is still free to move away from this maximum position and return to another point rest.

The hydraulic ankle assembly 118 uniquely incorporates a valve locking system that, when activated, allows hydraulic fluid to circulate until the device reaches its maximum dorsi flexion position, at which it becomes locked in this maximum position. Plantarflexion is prevented when the lock is activated. The hydraulic ankle assembly 118 remains locked until a valve is manually opened allowing the piston to move in the hydraulic cylinder. The normal operating position for the hydraulic ankle assembly 118 is in the unlocked position such that the foot has a full range of motion without restriction. The plantar flexion hydraulic lock helps amputees while driving by preventing plantarflexion which prevents the foot from unexpectedly interfering with the pedals. The plantar flexion hydraulic lock not only provides added safety but is also more natural as opposed to having a prosthesis that can rotate freely from the plantar flexion position to a dorsiflexion position.

At least some known prosthetic feet have included hydraulic locks to adjust the rotational position of the pyramid adapter so that the foot can be adjusted to accommodate for different shoe heel heights. However, these designs are solely used for heel height adjustment and not to articulate during gait, thus they do not offer the user with any of the inherent benefits of a hydraulic ankle/foot. The hydraulic ankle assembly 118 described herein allows for not only a lock-in-place condition but may also be placed in an open normal hydraulic condition affording the amputee with all the benefits of a hydraulic ankle.

Although volume compensators are common in industrial hydraulic systems, at least some known hydraulic ankles do not have built-in volume compensators or accumulators. Lack of fluid compensation can greatly hinder the function of the hydraulic ankle. In extreme conditions, the hydraulic fluid will either contract or expand, and fluid expansion results in high pressures and leakage. Lip seals are designed for pressure on one side of the seal, and fluid contraction results in pressure on the wrong side of the seal such that air may enter the hydraulic system. The constant pressure provided by a volume compensator inhibits cavitation of the hydraulic fluid. Cavitation creates heat and changes the viscosity of the hydraulic fluid, which affects hydraulic performance. The thermal expansion or contraction of the fluid volume can create numerous issues with a hydraulic ankle system, i.e.: air pockets can form in the fluid which hinder responsiveness. The system can over-pressurize causing seals to leak. In addition the hydraulic fluid acts as a lubricant for the shaft and seals and hence there is a very slow but consistent loss of fluid during use which can eventually result in hydraulic malfunction if the lost fluid is not replenished.

The hydraulic ankle assembly 118 described herein includes a nitrogen-filled volume compensator to enable pressurization of the fluid in the hydraulic system. The use of a chamber that is pressurized with an inert gas which contains little or no oxygen reduces the degradation of elastomeric seals and the pressure of nitrogen is not as sensitive to temperature as air.

The base 186 has a first side 192 and a second side 194. The first side 192 is sized and shaped to correspond to a shape of the first spring member 150 such that the posterior end of the first side 192 is arranged substantially flush with the first spring member 150. The second side 194 is sized and shaped to accommodate three sets of bore holes that attach the base 186 to the first spring member 150, the hydraulic cylinder 188, and the prosthetic adapter 190. Specifically, the base 186 defines a first set of bore holes 196a, 196b, a second set of bore holes 198a, 198b, and a third set of bore holes 200a, 200b. The first set of bore holes 196a, 196b are configured to receive fasteners 130a, 130b that fasten the base 186 to the first spring member 150. The second set of bore holes 198a, 198b are configured to receive a portion of the hydraulic cylinder 188 to maintain a position of the hydraulic cylinder 188 while allowing the hydraulic cylinder 188 to rotating relative to the base 186. Similarly, the third set of bore holes 200a, 200b are configured to receive a portion of the prosthetic adapter 190 to maintain a position of the prosthetic adapter 190 while allowing the prosthetic adapter 190 to rotating relative to the base 186. The base 186 is a monolithic and rigid part which does not function as a spring and exhibits no appreciable deflection or deformation during use and is made of a lightweight metal, for example aluminum, magnesium or titanium.

The prosthetic adapter 190 defines a bore 202 configured to receive the pyramid connector 132, a cavity 204 configured to receive a portion of the hydraulic cylinder 188, a fourth set of bore holes 206 configured to receive a piston fastener 208, and a base bore 210 configured to receive a base fastener 212. The prosthetic adapter 190 is sized and shaped to accommodate the pyramid connector 132, the cavity 204, the fourth set of bore holes 206, the piston fastener 208, the base bore 210, and the base fastener 212. Specifically, the prosthetic adapter 190 includes a first portion or bulbous portion 214 and a second portion or tapered portion 216. The bulbous portion 214 defines the cavity 204 and is bulbous to enable the cavity 204 to be voluminous enough to receive a portion of the hydraulic cylinder 188. Additionally, the bulbous portion 214 is large enough to define the bore 202 and the fourth set of bore holes 206 to accommodate the pyramid connector 132 and the piston fastener 208. The tapered portion 216 is smaller than the bulbous portion 214 such that the taper portion 216 is received between the third set of bore holes 200a, 200b of the base 186. The base fastener 212 extends between the third set of bore holes 200a, 200b and through the base bore 210 to attach the prosthetic adapter 190 to the base 186.

The hydraulic cylinder 188 includes a body 218 and a piston assembly 220 at least partially positioned with the body 218. The piston assembly 220 is configured to slide within the body 218 to resist and/or dampen rotation of the hydraulic ankle assembly 118. The piston assembly 220 includes a shaft 222 and a piston 224 attached to the shaft 222. The shaft defines a first bore hole 226 and the body defines a second bore hole 228. The first bore hole 226 receives the piston fastener 208 and enables the hydraulic cylinder 188 and the prosthetic adapter 190 to rotate relative to each other. The second bore hole 228 receives a piston body fastener 230 and enables the hydraulic cylinder 188 and the base 186 to rotate relative to each other.

The body 218 defines a plurality of body cavities 232 and a plurality of channels 234 that are configured to contain and channel a hydraulic fluid to dampen or prevent rotation of the hydraulic ankle assembly 118. Specifically, the body 218 defines a pressurization cavity 236, a piston cavity 238, a plantar valve cavity 240, a dorsi valve cavity 242, and a plantarflexion lock spool valve cavity 244. The body 218 further defines a pressurization channel 246, a first piston cavity channel 248, a second piston cavity channel 250, a dorsi flexion adjustment channel 252, and a plantar flexion adjustment channel 254. The body 218 may further define a accumulator port 256. The accumulator port 256 is designed as a hydraulic resistor which allows the accumulator to maintain fluid volume in the hydraulic circuit(s) yet minimizes pressure cycling in the accumulator. The fluid leaks through the gap between the male and female thread of the hydraulic body (218) and the accumulator cap (291), and this tolerance gap acts as an orifice. An alternate approach is to utilize an orifice, but orifices are relatively expensive due to the hard and inert materials required and the difficulty of drilling very small holes into such hard materials. Diamond, ruby, or sapphire orifices are frequently required to resist cavitation induced erosion and corrosion. Using threads to act as an orifice reduces the number of parts required, reducing cost and simplifying the hydraulic design. Cycling of the accumulator with every step results in wear of the accumulator piston seal and creates heat. Changes in fluid temperature result in changes in hydraulic resistance, and high fluid temperatures increase fluid and seal degradation rates. Minimizing heat generation is an important design consideration for a hydraulic ankle.

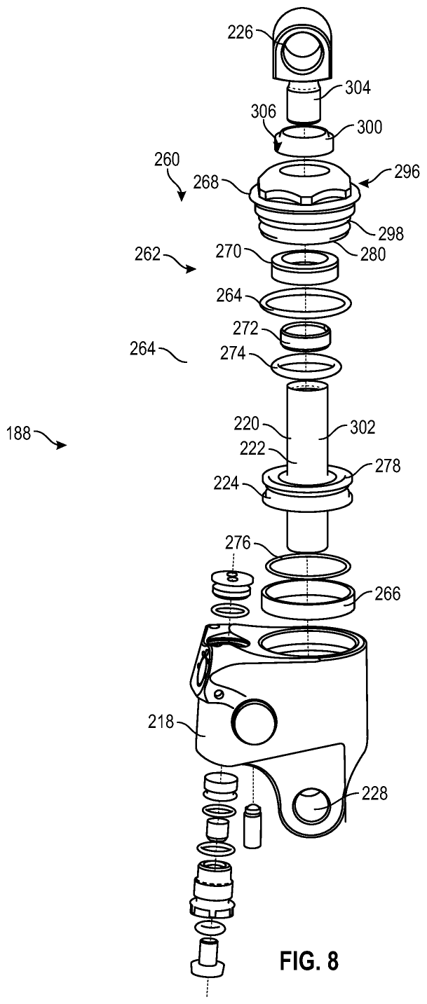

The hydraulic cylinder 188 also includes a plurality of wear rings or bushings 260 and seals 262. Specifically, the hydraulic cylinder 188 includes a shaft guide bushing 272 circumscribing the shaft 222 and positioned within the body 218 that protects and guides the shaft 222 within the body 218 and a piston guide bushing 266 and piston seal 276 circumscribing a portion of the piston 224 and positioned within the piston cavity 238 that protects and guides the piston 224 within the piston cavity 238. The hydraulic cylinder 188 also includes a first shaft wiper/seal 300, a second shaft seal 270, and a third shaft seal (not shown in FIG. 8) all circumscribing the shaft 222 and configured to prevent hydraulic fluid from leaking out of the hydraulic cylinder 188.

The hydraulic cylinder 188 further includes a dorsiflexion stop bumper 274 within the piston cavity 238 that improves the transition between the hydraulic resistance and spring resistance created by the carbon foot spring elements. The dorsiflexion stop bumper 274 is positioned on a surface 278 of the piston 224 and the dorsiflexion stop 274 contacts surface 280 of the piston cavity 238. The dorsiflexion stop 274 is made of a soft, elastic material that prevents or mitigates the impact of the piston 222 on the surface 280 of the piston cavity 238. Thus, the dorsiflexion stop 274 improves the transition between the hydraulic resistance and spring resistance created by the carbon foot spring elements.

The pressurization cavity 236 includes a cap 282, a plug 284, a pressurization piston 286, and a piston seal 288 circumscribing the pressurization piston 286. The pressurization cavity 236 defines an opening 290 and the cap 282 is removably positioned within the opening 290 and the plug 284 may be permanently positioned within the opening 290. The pressurization piston 286 separates the pressurization cavity 236 into a hydraulic fluid portion 292 and an inert gas portion 294. The hydraulic fluid portion 292 contains hydraulic fluid and is in fluid communication with the piston cavity 238, the plantar valve cavity 240, the dorsi valve cavity 242, and the plantarflexion lock spool valve cavity 244. The plug 284 is formed of an elastic material such as, but not limited to, rubber. The inert gas portion 294 contains an inert gas such as, but not limited to, nitrogen. The inert gas portion 294 is configured to pressurize the hydraulic fluid. Specifically, a user inserts a needle (not shown) of a syringe (not shown) through the plug 284 and injects the inert gas into the inert gas portion 294, increasing the pressure of the inert gas portion 294. The increased pressure of the inert gas portion 294 presses on the pressurization piston 286 which increases the pressure of the hydraulic fluid within the hydraulic cylinder 188. The pressurization channel 246 transmits the increased pressure to hydraulic fluid in the channels 234 and the body cavities 232.

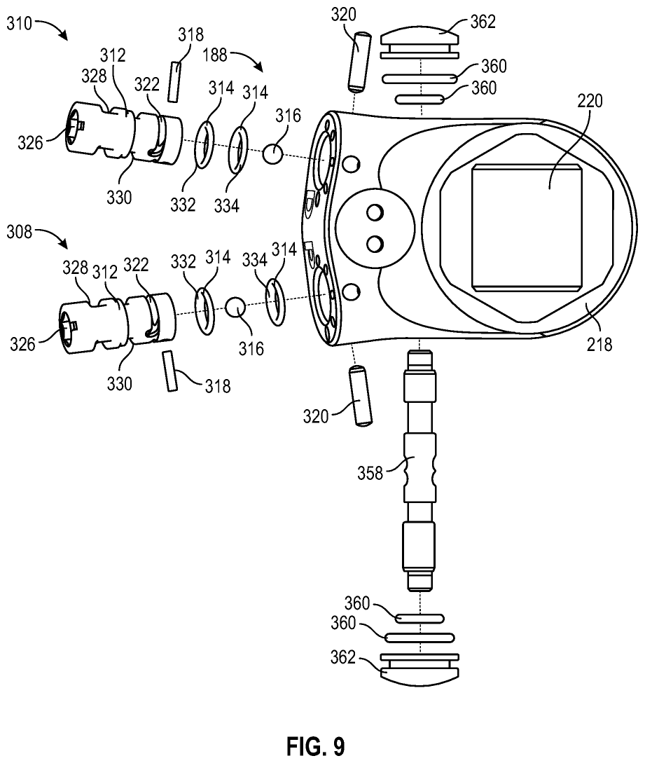

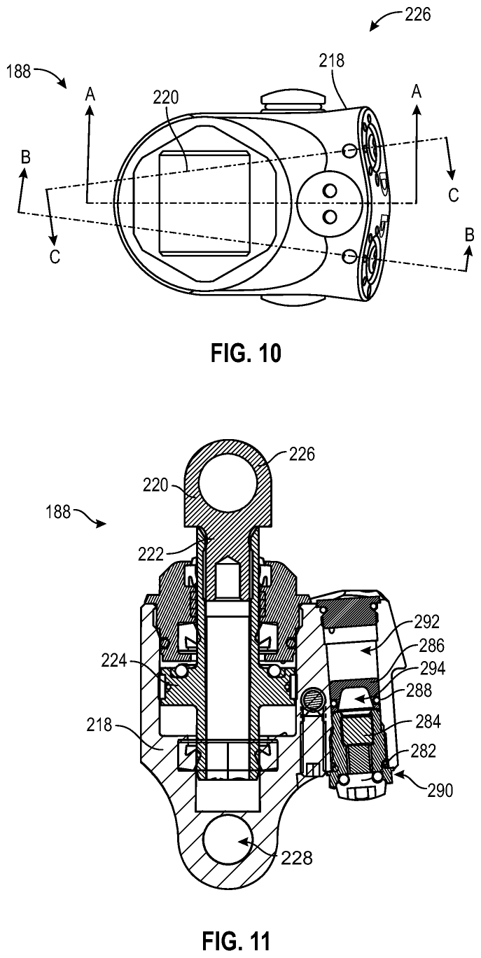

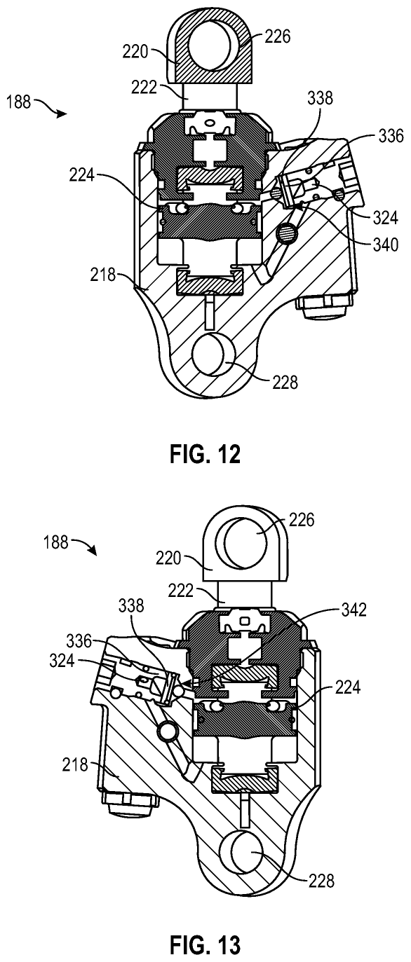

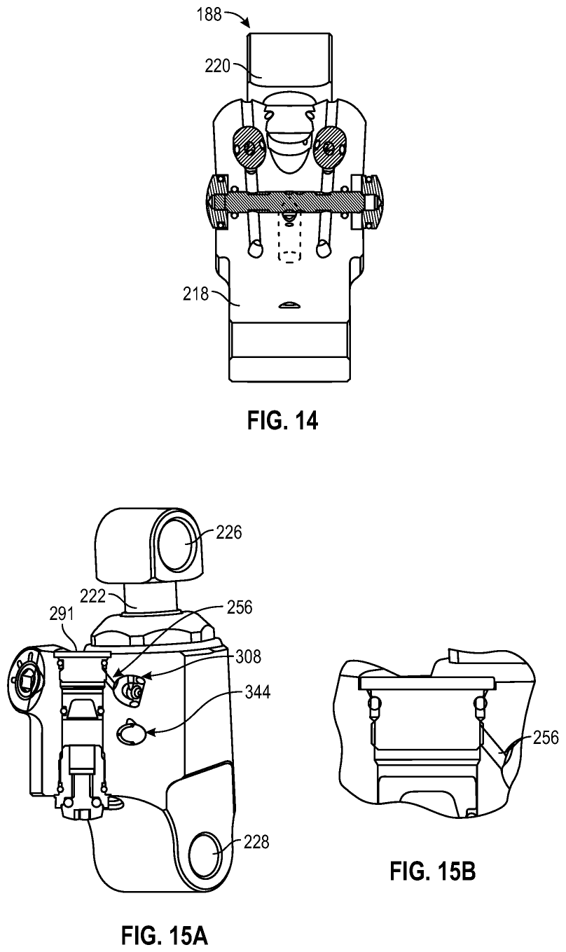

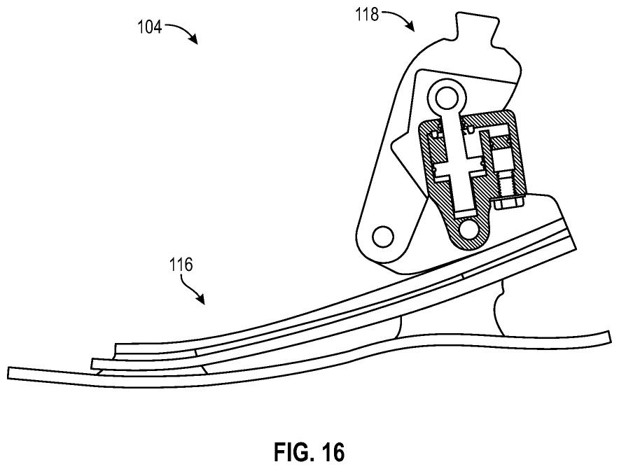

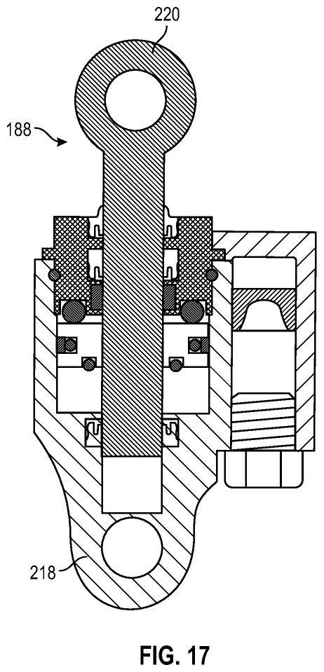

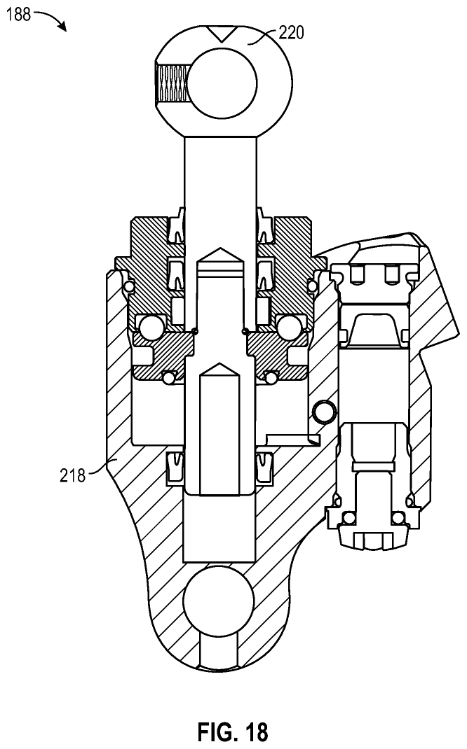

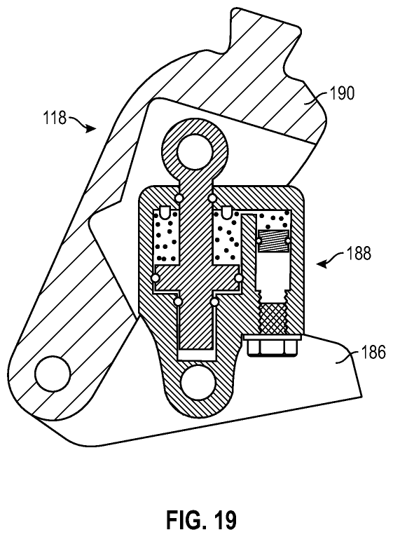

The hydraulic cylinder 188 further includes a cap 296 including threading 268, a cap seal 264 circumscribing a portion of the threading 298. The shaft 222 includes a first portion 302 screwed into a second portion 304. The hydraulic cylinder 188 is assembled by positioning the wear rings 260 and the seals 262 within the hydraulic cylinder 188 as shown in FIGS. 8-15. The first portion 302 is inserted into a bore 306 of the cap 296 and the second portion 304 is screwed into the first portion 302. The cap 296 is then screwed into the body 218 such that the piston assembly 220 is positioned within the within the hydraulic cylinder 188 as shown in FIGS. 17-23.

A plantarflexion adjustment valve 308 is positioned in the plantarflexion valve cavity 240 and a dorsiflexion adjustment valve 310 is positioned in the dorsi valve cavity 242. The plantarflexion adjustment valve 308 and the dorsiflexion adjustment valve 310 each include a valve body 312, at least one seal 314, a ball stock 316, a ball stock retention pin 318, and a valve retention pin 320. The valve body 312 define helical threading 322, an internal cavity 324, a screw head 326, a first side slot 328, and a second side slot 330. The valve body 312 is positioned in the plantar valve cavity 240 or the dorsi valve cavity 242 such that the screw head 326 faces out of the plantar valve cavity 240 or the dorsi valve cavity 242. In the illustrated embodiment, the screw head 326 includes a hexagonal recess. In alternative embodiments, the screw head 326 may include any type of screw head that enables the plantarflexion adjustment valve 308 or the dorsiflexion adjustment valve 310 to operate as described herein. Additionally, the valve body 312 is positioned in the plantar valve cavity 240 or the dorsi valve cavity 242 such that the helical threading 322 is positioned within and in flow communication with the plantar flexion adjustment channel 254 and the dorsi flexion adjustment channel 252.

Additionally, the valve retention pin 320 is inserted into the body 218 such that the valve retention pin 320 is partially positioned in the plantar valve cavity 240 or the dorsi valve cavity 242 and within the first side slot 328 such that the valve body 312 is rotatably maintained in the plantar valve cavity 240 or the dorsi valve cavity 242, and the first side slot in combination with pin 320, limits the rotational range of valves 308 and 310. The at least one seal 314 includes a first seal 332 positioned in the second side slot 330 and a second seal 334 positioned at an end of the valve body 312. The ball stock retention pin 318 is positioned within the internal cavity 324 and the ball stock 316 is positioned on either side of the ball stock retention pin 318 such that the ball stock 316 defines a check valve. Specifically, the ball stock 316 is positioned on a first side 336 of the ball stock retention pin 318 to define a first check valve 340 and the ball stock 316 is positioned on a second side 338 of the ball stock retention pin 318 and within the internal cavity 324 to define a second check valve 342. The valve body 312 is rotated to increase or decrease the length of the helical threading 322 the hydraulic fluid flows through to increase or decrease the pressure drop through the plantarflexion adjustment valve 308 and the dorsiflexion adjustment valve 310. The helical threading 322 has a variable depth and the pressure drop is adjusted by both increasing the flow path distance and altering the cross-sectional area of the flow path.

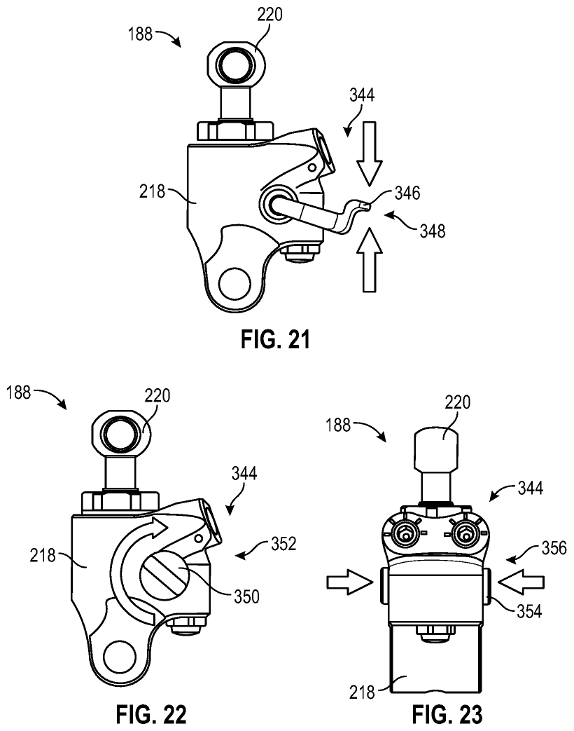

A plantarflexion lock spool valve 344 is positioned in the plantarflexion lock spool valve cavity 244 and is configured to prevent plantarflexion of the ankle such that when the ankle reaches maximum dorsiflexion it will be locked in the maximum dorsiflexion position. In the illustrated embodiment, the plantarflexion lock spool valve 344 is a manual hydraulic lock including at least one of a push button spool valve, rotatable knob spool valve, and a lever spool valve. Specifically, as shown in FIGS. 21-23, the plantarflexion lock spool valve 344 may include a lever 346 that actuates a lever spool valve 348, a rotatable knob 350 that actuates a rotatable knob spool valve 352, and/or at least one push button 354 that actuates a push button spool valve 356.

The plantarflexion lock spool valve 344 includes a valve body 358, at least one seal 360, and at least one end 362. The valve body 358 is positioned within the body 218 such that the valve body 358 is positioned within at least one of the channels 234 to prevent flow of the hydraulic fluid through the channels 234. Specifically, in the illustrated embodiment, the valve body 358 is positioned within the second piston cavity channel 250. The seals 360 circumscribe the valve body 358 and the ends 362 either include or are attached to the lever 346, the rotatable knob 350, and the push button 354.

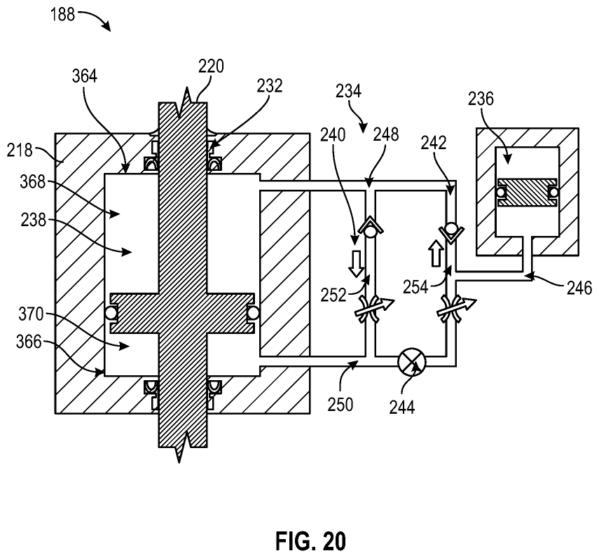

FIG. 20 illustrates a flow diagram of the body cavities 232, the channels 234, and the valves 308, 310, 340, 342, and 344. As described below, the patient actuates the hydraulic cylinder 188 by walking such that the piston assembly 220 is shifted toward a first end 364 of the piston cavity 238 or a second end 366 of the piston cavity 238. The piston 224 divides the piston cavity 238 into a first cavity 368 and a second cavity 370. As the piston 224 moves through the piston cavity 238, hydraulic fluid is channeled from one of the first cavity 368 and the second cavity 370 through the channels 234.

Specifically, during dorsiflexion, the piston 224 shifts up toward the first end 364 of the piston cavity 238 such that a volume of the first cavity 368 is reduced and the hydraulic fluid within the first cavity 368 is displaced through the channels 234 into the second cavity 370. More specifically, in the illustrated embodiment, the hydraulic fluid within the first cavity 368 is channeled through the first piston cavity channel 248, the dorsi flexion adjustment channel 252, and the second piston cavity channel 250 and into the second cavity 370. The dorsiflexion adjustment valve 310 is rotated to increase or decrease the pressure drop through the dorsiflexion adjustment valve 310 to control the dampening effect of the hydraulic cylinder 188 on the dorsiflex movement. The second check valve 342 prevents the hydraulic fluid from flowing through the plantar flexion adjustment channel 254.

During plantarflexion, the piston 224 shifts down toward the second end 366 of the piston cavity 238 such that a volume of the second cavity 370 is reduced and the hydraulic fluid within the second cavity 370 is displaced through the channels 234 into the first cavity 368. More specifically, in the illustrated embodiment, the hydraulic fluid within the second cavity 370 is channeled through the second piston cavity channel 250, the plantar flexion adjustment channel 254, and the first piston cavity channel 248 and into the first cavity 368. The plantarflexion adjustment valve 308 is rotated to increase or decrease the pressure drop through the plantarflexion adjustment valve 308 to control the dampening effect of the hydraulic cylinder 188 on the plantarflexion movement. The first check valve 340 prevents the hydraulic fluid from flowing through the dorsi flexion adjustment channel 252.

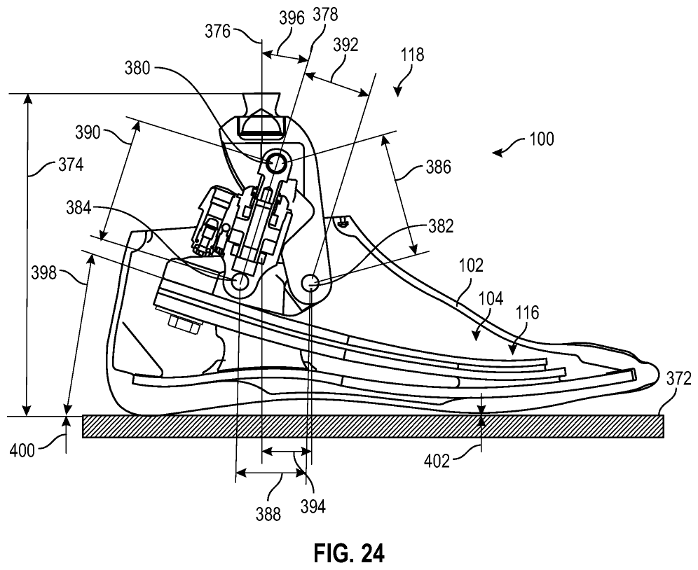

FIG. 24 illustrates the prosthetic foot system 100 positioned on a flat, horizontal ground surface 372 in a neutral stance such that a pyramid angle 374 between the top surface of the pyramid connector 132 is 0°. As shown in FIG. 24, the pyramid connector 132 defines a pyramid connector axis 376 through a middle of the pyramid connector 132 which is oriented vertically. The piston assembly 220 defines a piston axis 378 through a middle of the shaft 222.

The piston fastener 208 defines a first pivot point 380 extending through a middle of the piston fastener 208, the base fastener 212 defines a second pivot point 382 extending through a middle of the base fastener 212, and the piston body fastener 230 defines a third pivot point 384 extending through a middle of the piston body fastener 230. A first pivot distance 386 is defined as the distance between the first pivot point 380 and the second pivot point 382. A second pivot distance 388 is defined as the distance between the second pivot point 382 and the third pivot point 384. A third pivot distance 390 is defined as the distance between the first pivot point 380 and the third pivot point 384.

Additionally, a moment arm 392 is defined as the distance between the second pivot point 382 and the piston axis 378. A pyramid axis distance 394 is defined as the distance between the second pivot point 382 and the pyramid connector axis 376. An axis angle 396 is defined as the angle between the pyramid connector axis 376 and the piston axis 378. A top spring angle 398 is defined as the angle between the flat surface 372 and the first spring member 150. A heel distance 400 is defined as the distance between the flat surface 372 and a heel portion of the footshell 102 and a forefoot distance 402 is defined as the distance between the flat surface 372 and a forefoot portion of the footshell 102.

The pyramid axis of a prosthetic foot is typically located at between 25% to 35% of the foot length measured from the heel end of the footshell. As shown in FIG. 28, the center of mass is located between 65 and 100 mm anterior to the posterior most end of the foot shell depending on foot size, or at ⅓ of the foot length±10 mm. Some adjustment to the location of a prosthetic foot based on the effective spring stiffness of the prosthetic foot may be made by a prosthetist.

In the illustrated embodiment, the first pivot distance 386, the second pivot distance 388, and the third pivot distance 390 define a force triangle that distributes the weight of the user and defines an axis of rotation of the hydraulic ankle assembly 118. In the illustrated embodiment, the axis of rotation of the hydraulic ankle assembly 118, more specifically the axis of rotation of the base and foot spring assembly is positioned forward of the pyramid connector 132 and below and in line with the center of mass of the user. The rotation axis of the foot is forward of the pyramid axis. Therefore, the center of mass of the user is directly above the rotation axis when standing, allowing users to stand without significant movement of the hydraulic ankle assembly 118. Thus, the above-described geometry of the hydraulic ankle assembly 118 also minimizes impact when the ankle reaches the end of its hydraulic range in the dorsiflexion direction (the dorsiflexion stop).

In the embodiment illustrated in FIG. 24, the first pivot distance 386 is about 50 millimeters (mm) to about 60 mm or about 58.8 mm, the second pivot distance 388 is about 25 mm to about 40 mm or about 33.0 mm, the third pivot distance 390 is about 50 mm to about 60 mm or about 58.6 mm, the moment arm 392 is about 25 mm to about 40 mm or about 31.7 mm, the pyramid axis distance 394 is about 19 mm to about 26 mm or about 23.0 mm, the axis angle 396 is about 15° to about 20° or about 17.1°, the top spring angle is about 15° to about 20° or about 18.2°, and the heel distance 400 and the forefoot distance 402 are both about 0 mm, indicating the foot, including the footshell, is flat on a horizontal ground surface.

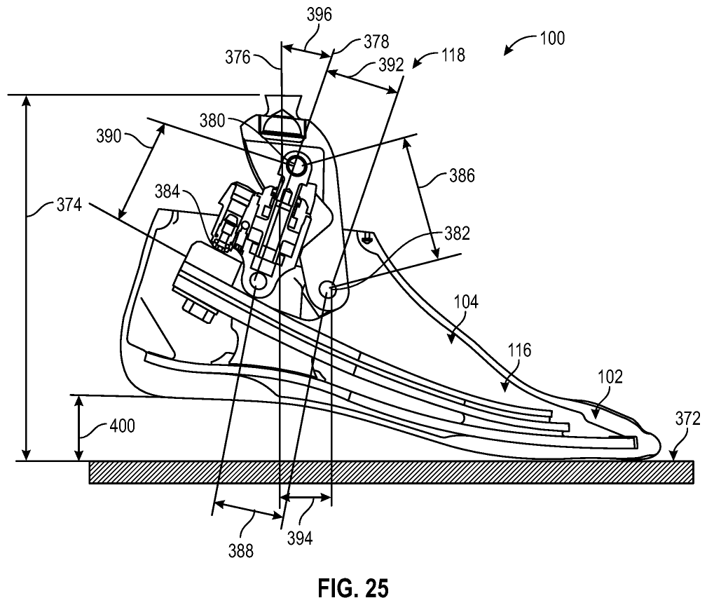

FIG. 25 illustrates the prosthetic foot system 100 positioned on the flat surface 372 at maximum plantarflexion such that the pyramid angle 374 of the pyramid connector 132 is 0°. In the embodiment illustrated in FIG. 25, the first pivot distance 386 is about 50 millimeters (mm) to about 60 mm or about 58.8 mm, the second pivot distance 388 is about 25 mm to about 40 mm or about 33.0 mm, the third pivot distance 390 is about 50 mm to about 60 mm or about 52.5 mm, the moment arm 392 is about 25 mm to about 40 mm or about 32.8 mm, the pyramid axis distance 394 is about 19 mm to about 26 mm or about 23.0 mm, the axis angle 396 is about 15° to about 20° or about 18.3°, the top spring angle is about 20° to about 30° or about 29.1°, and the heel distance 400 is about 30 mm to about 40 mm or about 30.23 mm.

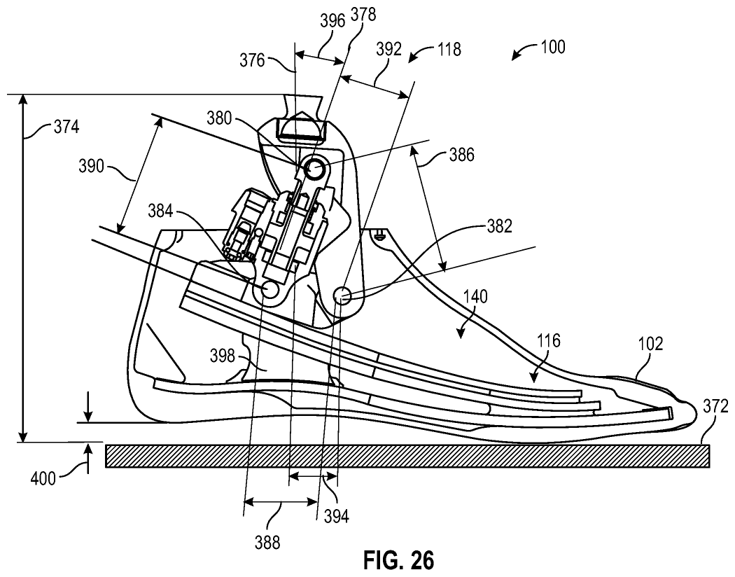

FIG. 26 illustrates the prosthetic foot system 100 positioned on the flat surface 372 when the prosthetic foot system 100 is arranged in a shoe (not shown) such that the pyramid angle 374 of the pyramid connector 132 is 2°. The heel distance 400 is a typical shoe height of about 5 mm to about 20 mm or about 10 mm. In the embodiment illustrated in FIG. 26, the first pivot distance 386 is about 50 millimeters (mm) to about 60 mm or about 58.8 mm, the second pivot distance 388 is about 25 mm to about 40 mm or about 33.0 mm, the third pivot distance 390 is about 50 mm to about 60 mm or about 57.5 mm, the moment arm 392 is about 25 mm to about 40 mm or about 32.0 mm, the pyramid axis distance 394 is about 19 mm to about 26 mm or about 23.0 mm, the axis angle 396 is about 15° to about 20° or about 17.4°, and the top spring angle is about 20° to about 30° or about 22.3°.

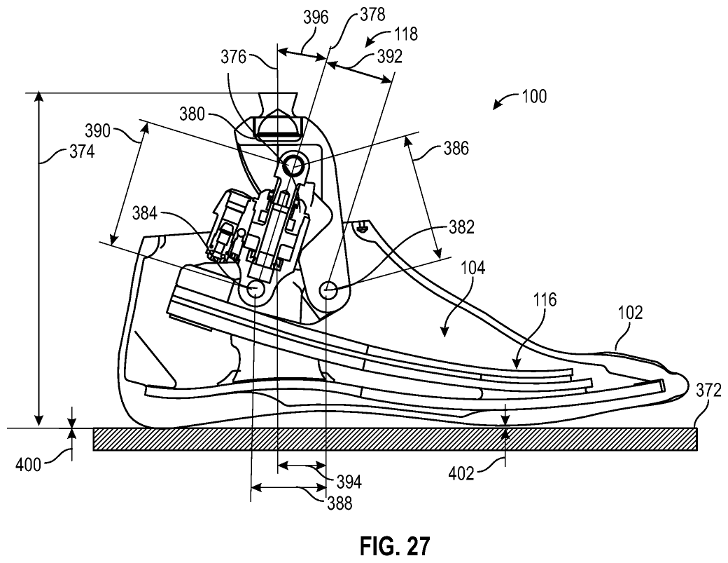

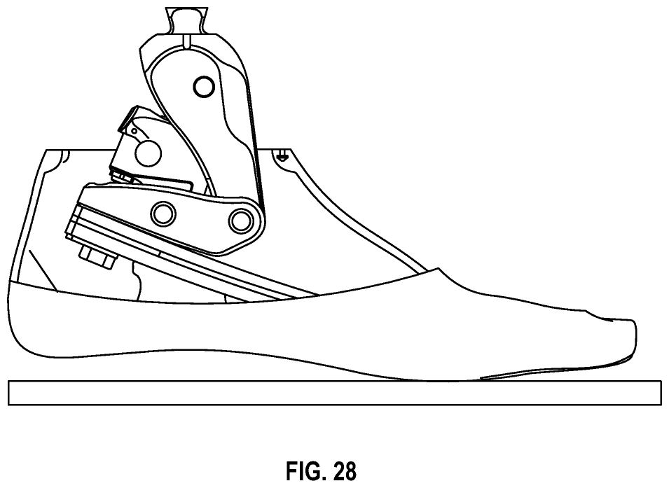

FIG. 27 illustrates the prosthetic foot system 100 positioned on the flat surface 372 at maximum dorsiflexion such that the pyramid angle 374 of the pyramid connector 132 is 0°. In the embodiment illustrated in FIG. 27, the first pivot distance 386 is about 50 millimeters (mm) to about 60 mm or about 58.8 mm, the second pivot distance 388 is about 25 mm to about 40 mm or about 33.0 mm, the third pivot distance 390 is about 50 mm to about 60 mm or about 58.6 mm, the moment arm 392 is about 25 mm to about 40 mm or about 31.7 mm, the pyramid axis distance 394 is about 19 mm to about 26 mm or about 23.0 mm, the axis angle 396 is about 15° to about 20° or about 17.1°, the top spring angle is about 10° to about 20° or about 18.2°, the heel distance 400 is about 0 mm, and the forefoot distance 402 is about 0 mm to about 10 mm or about 1.13 mm.

The above-described geometry of the hydraulic ankle assembly 118 enables plantarflexion motion at an ankle joint, which allows the metatarsophalangeal (i.e. ball of foot) area of the foot to achieve contact with the ground earlier in the gait cycle. The ball of foot/wide part of the foot provides stability during the gait cycle. The above-described geometry of the hydraulic ankle assembly 118 also enables a small amount of dorsiflexion, relative to a standing position, which results in reduced and adjustable hydraulically controlled resistance to tibial progression during the portion of the gait cycle between foot flat and midstance when the rotational range of the hydraulic ankle assembly reaches a dorsiflexion limit. When an amputees' center of mass is directly above the shin and the shin is vertical, the amputee does not have much leverage over the lever arm created by the forefoot of a prosthetic foot. The above-described geometry of the hydraulic ankle assembly 118 further enables clearance during swing phase which reduces stumbling and falling. Because the rotation around the hydraulic ankle assembly 118 is dampened by hydraulic resistance, the foot portion stays in a dorsiflexed position during swing phase until the heel makes contact with the ground and plantarflexion begins.

For example, the rotation axis of the foot is forward of the pyramid axis. Therefore, the center of mass (COM) of the amputee is directly above the rotation axis when standing, allowing amputees to stand without significant movement of the hydraulic ankle assembly 118. Thus, the above-described geometry of the hydraulic ankle assembly 118 also minimizes impact when the ankle reaches the end of its hydraulic range in the dorsiflexion direction (the dorsiflexion stop).

The hydraulic ankle assembly 118 also includes the dorsiflexion stop 274 that reduces or eliminates an impact at the end of the dorsiflexion travel. As the hydraulic ankle assembly 118 dorsi flexes, it reaches the end of its hydraulic range-of-motion and the hydraulic action abruptly stops. At this point the flex of the spring assembly 116 takes over and starts to bend. This transition can be abrupt and not comfortable for the amputee. The dorsiflexion stop 274 gradually squeezes as the hydraulic ankle assembly 118 reaches this transition point, allowing a smooth transition from the hydraulic function to the flexing function of the spring assembly 116. Additionally, the spring assembly 116 may be low-profile disc springs that would respond is a similar way as the elastomeric stop and provide a smooth transition between the hydraulic function and the carbon spring function of the hydraulic ankle assembly 118 and spring assembly 116.

Moreover, the geometry of the hydraulic ankle assembly 118 described herein may include a greater distance between the pivot axes 382 and 384 on the base 186 compared to other hydraulic ankle/feet. Increased pivot distance increases the distance between the cylinder axis and the foot pivot point (the moment arm 392), which reduces the forces on the hydraulic cylinder 188 and results in reduced stresses and strains in the hydraulic cylinder 188 and also allows for either decreased hydraulic pressure or a smaller cylinder and piston diameter at equivalent pressure. Reducing forces and maximum hydraulic pressures increases reliability and reduces weight.

The hydraulic ankle assembly 118 described herein utilizes a straight hydraulic cylinder 188 including three pivot axes to eliminate transverse loads on the shaft 222. At least some known hydraulic ankle assemblies may include only two pivot axes, resulting in transverse load on the cylinder and shaft which is accommodated with large clearances between parts which may result in fluid leakage as seals wear. At least some known hydraulic ankle assemblies may include an extra link added the end of the shaft to achieve three pivot axes, increasing the part count, weight, and cost. The three pivot axes design, without an additional link, disclosed in the hydraulic ankle assembly 118 is an improvement over current designs.

At least some known passive hydraulic systems may not by means of a hydraulic lock. The limiting range of motion in current designs occur when the hydraulic piston reaches its maximum position in the cylinder. Once the piston reaches this maximum position (whether in the dorsi flexion or plantar flexion positions) it is still free to move away from this maximum position and return to another point rest.

The hydraulic ankle assembly 118 uniquely incorporates the plantarflexion lock spool valve 344 that, when activated, allows hydraulic fluid to circulate until the device reaches its maximum dorsi flexion position, at which it becomes locked in this maximum position. Plantarflexion is prevented when the plantarflexion lock spool valve 344 is activated. The hydraulic ankle assembly 118 remains locked until the plantarflexion lock spool valve 344 is manually opened allowing the piston to move in the hydraulic cylinder. The normal operating position for the hydraulic ankle assembly 118 is in the unlocked position such that the foot has a full range of motion without restriction. The plantarflexion lock spool valve 344 helps amputees while driving by preventing plantarflexion which prevents the foot from unexpectedly interfering with the pedals. The plantarflexion lock spool valve 344 provides added safety for driving and unique tasks such as steadying oneself in awkward footing situations, and provides a repeatable locked position. An articulating ankle such as a hydraulic ankle with a locking feature which locks movement in both directions is difficult to lock in a desired position. The foot must be placed in the desired position and then the locking mechanism activated. Activating the locking feature without unintentionally altering position of the foot may be difficult and may require sitting. The plantarflexion lock disclosed allows a user to activate the lock while standing and then move the foot to the maximum dorsiflexion position, which prevents movement of the ankle until the locking mechanism is de-activated.

At least some known prosthetic feet have included hydraulic locks to adjust the rotational position so that the foot can be adjusted to accommodate for different shoe heel heights. However, these designs are solely used for heel height adjustment and not to articulate during gait, thus they do not offer the user with any of the inherent benefits of a hydraulic ankle/foot. The hydraulic ankle assembly 118 described herein allows for not only a lock-in-place condition but may also be placed in an open normal hydraulic condition affording the amputee with all the benefits of a hydraulic ankle.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A prosthetic ankle assembly comprising:
a prosthetic adapter;
a hydraulic cylinder rotatably attached to the prosthetic adapter to define a first pivot point, the hydraulic cylinder configured to resist rotation of the hydraulic ankle assembly; and
a monolithic base attached to the prosthetic adapter to define a second pivot point and attached to the hydraulic cylinder to define a third pivot point, wherein the first pivot point and the second pivot point define a first pivot distance, the second pivot point and the third pivot point define a second pivot distance, and the first pivot point and the third pivot point define a third pivot distance; and
wherein the first pivot distance is greater than 30 millimeters (mm), the second pivot distance is greater than 30 mm, and the third pivot distance is greater than 30 mm.

2. The prosthetic ankle assembly of claim 1, wherein the second pivot distance is about 30 mm to about 35 mm.

3. The prosthetic ankle assembly of claim 2, wherein the first pivot distance is about 50 mm to about 60 mm.

4. The prosthetic ankle assembly of claim 3, wherein the third pivot distance is about 50 mm to about 60 mm.

5. The prosthetic ankle assembly of claim 1, wherein the hydraulic cylinder is a hydraulic cylinder configured to dampen rotation of the ankle assembly.

6. The prosthetic ankle assembly of claim 4, wherein the hydraulic cylinder is a hydraulic cylinder configured to dampen rotation of the ankle assembly.

7. The prosthetic ankle assembly of claim 1, wherein an axis of rotation is positioned forward of a pyramid connector axis and is configured to be in line with a center of mass of a user when the user is in a standing position

8. The prosthetic ankle assembly of claim 5, wherein an axis of rotation is positioned forward of a pyramid connector axis and is configured to be in line with a center of mass of a user when the user is in a standing position.

9. The prosthetic ankle assembly of claim 7, wherein the hydraulic cylinder comprises a plurality of cavities and channels configured to channel a hydraulic fluid and dampen rotation of the hydraulic ankle assembly and a plantarflexion lock valve positioned within at least one of the cavities and the channels; and
wherein the plantarflexion lock valve allows hydraulic fluid to circulate until the hydraulic ankle assembly reaches a maximum dorsiflexion position and locks at the maximum dorsiflexion position.

10. The prosthetic ankle assembly of claim 7, wherein the hydraulic cylinder comprises a plurality of cavities and channels configured to channel a hydraulic fluid and dampen rotation of the hydraulic ankle assembly including and accumulator and an orifice; and
wherein the orifice is comprised of a leak path through mating threads.

11. A prosthetic ankle assembly comprising:
a rigid base attached to at least one composite foot spring, the rigid base defining a foot pivot axis,
a prosthetic adapter rotatably attached to the rigid base, the prosthetic adapter comprising a prosthetic pyramid with a pyramid axis;
the foot pivot axis positioned forward of the pyramid connector axis;
a hydraulic cylinder defining an axis between rotatable connection points and attached to the prosthetic adapter and to the base;
wherein a moment arm distance between the hydraulic cylinder axis and the foot pivot axis is greater than 25 millimeters (mm).

12. The prosthetic ankle assembly of claim 11, wherein the moment arm distance is about 25 mm to about 40 mm.

13. The prosthetic ankle assembly of claim 12, wherein the moment arm distance is about 31.7 mm.

14. The prosthetic ankle assembly of claim 13, wherein the hydraulic cylinder comprises a hydraulic cylinder including a plurality of cavities and channels configured to channel a hydraulic fluid and dampen rotation of the ankle assembly.

15. The prosthetic ankle assembly of claim 14, wherein the hydraulic cylinder also comprises a plantarflexion lock valve positioned within at least one of the cavities and the channels; and
wherein the plantarflexion lock valve allows hydraulic fluid to circulate until the hydraulic ankle assembly reaches a maximum dorsiflexion position and locks at the maximum dorsiflexion position.

16. The prosthetic ankle assembly of claim 14, wherein the hydraulic cylinder comprises a plurality of cavities and channels configured to channel a hydraulic fluid and dampen rotation of the hydraulic ankle assembly including and accumulator and an orifice; and
wherein the orifice is comprised of a leak path through mating threads.

17. A prosthetic ankle assembly comprising:
a metal base configured to be attached to a foot spring assembly;
an hydraulic cylinder rotatably attached to the metal base and configured to control rotation of the prosthetic ankle assembly; and
a prosthetic adapter rotatably attached to the metal base and the hydraulic cylinder and configured to be attached to a prosthetic worn by a user, the prosthetic adapter comprising a pyramid connector that defines a pyramid connector axis,
wherein the metal base, a hydraulic cylinder, and the prosthetic adapter define a force triangle that defines an axis of rotation of the prosthetic ankle assembly, and
wherein the axis of rotation is positioned forward of the pyramid connector axis.

18. The prosthetic ankle assembly of claim 16, wherein the axis of rotation is configured to be in line with a center of mass of the user when the user is in a standing position.

19. The prosthetic ankle assembly of claim 17, wherein a moment arm distance between the hydraulic cylinder and a foot pivot axis is greater than 25 mm.

20. The prosthetic ankle assembly of claim 18, wherein the hydraulic cylinder includes a hydraulic cylinder configured to dampen rotation of the prosthetic ankle assembly.

21. The prosthetic ankle assembly of claim 19, wherein the hydraulic cylinder comprises a plurality of cavities and channels configured to channel a hydraulic fluid and dampen rotation of the prosthetic ankle assembly and a plantarflexion lock valve positioned within at least one of the cavities and the channels; and
wherein the plantarflexion lock valve allows hydraulic fluid to circulate until the prosthetic ankle assembly reaches a maximum dorsiflexion position and locks at the maximum dorsiflexion position.
